(19) United States
(12) Patent Application Publication
(10) Pub. No.: US 2016/0199083 A1
(43) Pub. Date: Jul. 14, 2016

(54) SURGICAL INSTRUMENT FOR ILIAC VEIN SURGERY

(71) Applicant: CATHOLIC UNIVERSITY INDUSTRY ACADEMIC COOPERATION FOUNDATION, Seoul (KR)

(72) Inventors: In-Sung Moon, Seoul (KR); Ji-Il Kim, Gyeonggi-do (KR); Sun-Cheol Park, Seoul (KR); Jeong-Kye Hwang, Seoul (KR)

(21) Appl. No.: 14/786,891

(22) PCT Filed: Dec. 5, 2013
(86) PCT No.: PCT/KR2013/011208

§ 371 Date: Mar. 14, 2016

(30) Foreign Application Priority Data
Apr. 25, 2013 (KR) 10-2013-0046230

(51) Int. Cl. A61B 17/28; A61B 17/29

(52) U.S. Cl. CPC A61B 17/2841; A61B 17/2909; A61B 2017/00778

(57) ABSTRACT

The present invention relates to a surgical instrument for an iliac vein surgery includes a pressurizing part for fixing an iliac vein, an inclined part inclinedly formed on the end of one side of the pressurizing part, a height control part, for controlling the height, formed on the end of the opposite side to the one side of the pressurizing part, and a handle part formed on the end of the height control part. The present invention has the effects of minimizing interference of a surgical instrument and the hands of an operator at the time of anastomosis during iliac vein surgery, and accordingly reducing operation time and costs.

### TECHNICAL FIELD

The present invention relates to a surgical instrument for an iliac vein surgery, which is used in an iliac vein clamp for venous anastomosis in kidney and pancreas transplantations.

### BACKGROUND ART

The surgery on the iliac vein is one of the common vascular procedures, such as kidney transplantation, pancreas transplantation, veno-venous bypass for unilateral iliac vein thrombosis (Palma-Dale Operation), hemostasis for traumatic internal organ damage in the pelvis, iatrogenic iliac vein injury in rectal cancer or gynecological cancer surgery, etc.

The surgical instrument of the present invention is used for this kind of operations and is mainly used in the high degree of difficulty and emergency surgery.

A bulldog clamp is used in a conventional vein surgery. However, two bulldog clamps are necessary for a complete hemostasis and the iliac vein is required to be exposed after complete dissection. In addition, the bulldog clamp does not sufficiently support and hold the iliac vein and suture materials are easily stuck to the bulldog clamp during the operation.

In addition, a conventional Satinsky clamp is used even though the iliac vein is not completely dissected, but a grip portion of the Satinsky clamp is placed at a high position during the operation since the Satinsky clamp is manufactured in the way that is suitable for a westerner's body rather than an easterner's body. As a result, the grip portion of the Satinsky clamp interferes with operator's view, the operation becomes difficult, and operation hours become longer.

### DISCLOSURE OF THE INVENTION

### Technical Problem

The present invention provides a surgical instrument for iliac vein surgery to reduce interference between a surgical instrument and an operator's hand during venous anastomosis in iliac vein surgery.

The present invention also provides a surgical instrument for iliac vein surgery in which a position and a rotation angle thereof are easily controlled by adjusting a height control part and a rotating part.

### Technical Solution

The present invention provides a surgical instrument for an iliac vein surgery, including a pressurizing part, an inclined part, a height control part, and a handle part. The pressurizing part fixes an iliac vein. The inclined part is disposed at an end of one side of the pressurizing part and inclined with respect to the pressurizing part. The height control part is disposed at an end of an opposite side to the one side of the pressurizing part to control a height thereof. The handle part is disposed at an end of the height control part.

The present invention also provides surgical instrument for an iliac vein surgery, including two pressurizing parts, an inclined part, a height control part, a handle part, and a rotation axis. The two pressurizing parts fix an iliac vein. The inclined part is disposed at each end of one side of the pressurizing parts and inclined with respect to the pressurizing parts. The height control part is disposed at each end of an opposite side to the one side of the pressurizing parts to control a height thereof. The handle part is disposed at an end of the height control part. The rotation axis is equipped with a contact portion of the height control part.

In an embodiment of the present invention, the height control part includes a lower control portion, an upper control portion, and a fixing part. The lower control portion has a predetermined height, includes an accommodating space defined therein, and includes screw-engaging holes formed through the lower control portion to be spaced apart from each other. The upper control portion is inserted into the accommodating space and provided with screw-engaging recesses formed through the upper control portion to be spaced apart from each other. The fixing part is spiral-engaged with the screw-engaging holes and the screw-engaging recesses.

In an embodiment of the present invention, the height control part includes a lower control portion, an upper control portion, and a fixing pin. The lower control portion has a predetermined height, includes an accommodating space defined therein, and includes lower thru-holes formed through the lower control portion to be spaced apart from each other. The upper control portion is inserted into the accommodating space and provided with upper thru-holes formed through the upper control portion to be spaced apart from each other. The fixing pin is insertion-coupled with the lower thru-holes and the upper thru-holes and includes a retaining ring disposed at an end of the fixing pin and bent.

In an embodiment of the present invention, the height control part includes a lower control portion, an upper control portion, and a plurality of control members. The lower control portion includes an accommodating space defined therein. The upper control portion is separated from the lower control portion and includes a coupling protrusion disposed at an end thereof. The control members are coupled between the lower control portion and the upper control portion and include a coupling protrusion disposed at a lower end of each control member and a coupling recess disposed at an upper end of each control member to be insertion-coupled with each other.

In an embodiment of the present invention, the handle part has a predetermined length and includes a circular ring disposed at an end thereof.

In an embodiment of the present invention, a rotating part is equipped with the height control part or the handle part. The height control part or the handle part, with which the rotating part is equipped, includes a first separating part and a second separating part separated from the first separating part.

In an embodiment of the present invention, the rotating part includes a rotation coupling hole and a rotation protrusion. The rotation coupling hole is formed in the first separating part and has a gear shape. The rotation protrusion is protrusion-formed in the second separating part and has a gear shape.

In an embodiment of the present invention, a thru-hole is formed through a center portion of each of the first and second separating parts, a fixing bolt is inserted into the thru-hole at one side of the thru-hole, and a fixing nut is coupled with the fixing bolt at an opposite side to the one side.

In an embodiment of the present invention, the rotating part includes an axis coupling hole and an axis coupling protrusion, which are respectively formed through centers of the first separating part and the second separating part for the insertion-coupling between the first and second separating parts. Pin coupling holes are formed through an end of the first separating part to be spaced apart from each other along a circumference of the first separating part. A rotation fixing pin, of which one end thereof is bent, is insertion-coupled to the pin coupling holes.

In an embodiment of the present invention, the rotating part includes an axis coupling hole and an axis coupling protrusion, which are respectively formed through centers of the first separating part and the second separating part for the insertion-coupling between the first and second separating parts. Insertion-coupling recesses are formed through an end of the first separating part to be spaced apart from each other along a circumference of the first separating part. An insertion-fixing member is formed along an outer surface of the second separating part, and the insertion-fixing member moves in a horizontal direction to be insertion-coupled to the insertion-coupling recesses of the first separating part.

### Advantageous Effects

According to the present invention, the interference between the surgical instrument and the operator's hands can be minimized at the time of anastomosis during iliac vein surgery, and accordingly the operation time and cost may be reduced.

In addition, the height and rotation angle of the surgical instrument for the iliac vein surgery may be easily controlled by operating the height control part and the rotating part depending on the condition of patient, and thus anyone may quickly respond depending on the situation.

### BRIEF DESCRIPTION OF THE DRAWINGS

FIG. 1 is a perspective view showing a surgical instrument for an iliac vein surgery according to the present invention.

FIGS. 2 and 3 are views of another embodiment of a surgical instrument for an iliac vein surgery according to the present invention.

FIGS. 4 and 5 are views of another embodiment of a surgical instrument for an iliac vein surgery according to the present invention.

FIGS. 6 to 8 are views showing a height control part of a surgical instrument for an iliac vein surgery according to the present invention.

FIGS. 9 to 11 are views showing a rotating part of a surgical instrument for an iliac vein surgery according to the present invention.

FIGS. 12 and 13 are views showing a state of use of the surgical instrument for an iliac vein surgery according to the present invention.

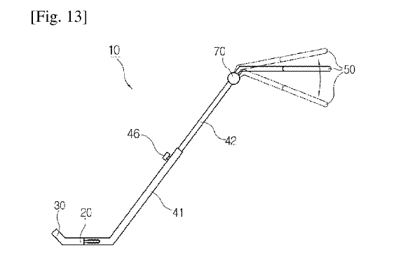

### BEST MODE FOR CARRYING OUT THE INVENTION

Hereinafter, exemplary embodiments of the present invention and a comparison example will be described in detail. However, it should be noted that the present invention is not limited to the following exemplary embodiments and may be implemented in various forms. Accordingly, the exemplary embodiments are provided only to disclose the present invention and let those skilled in the art know the category of the present invention.

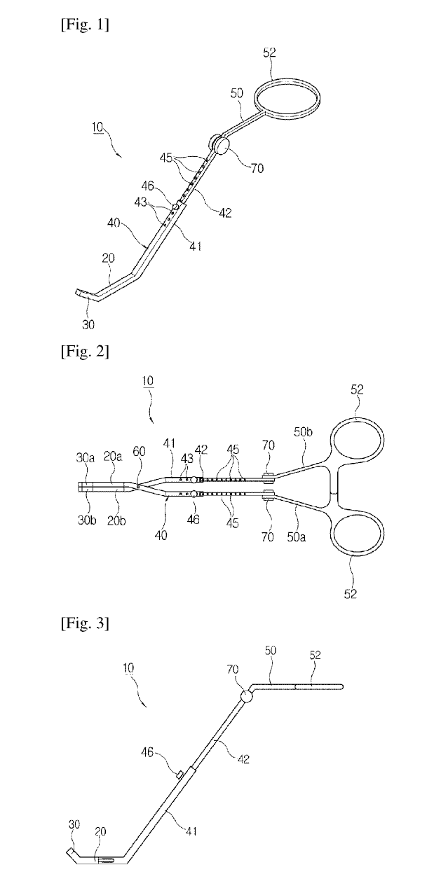

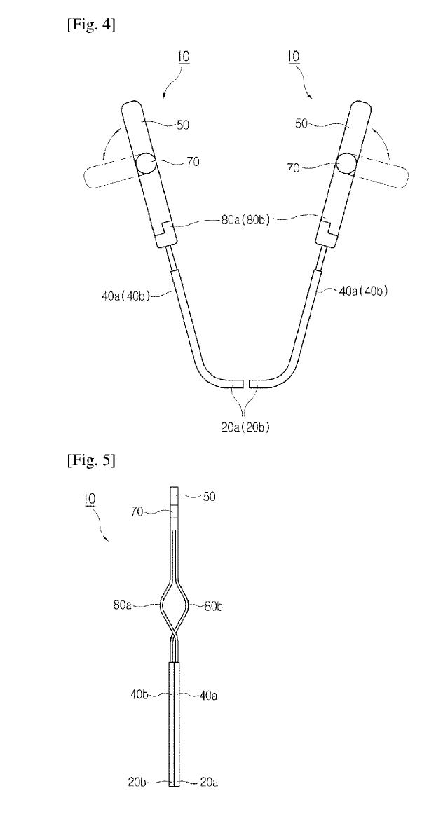

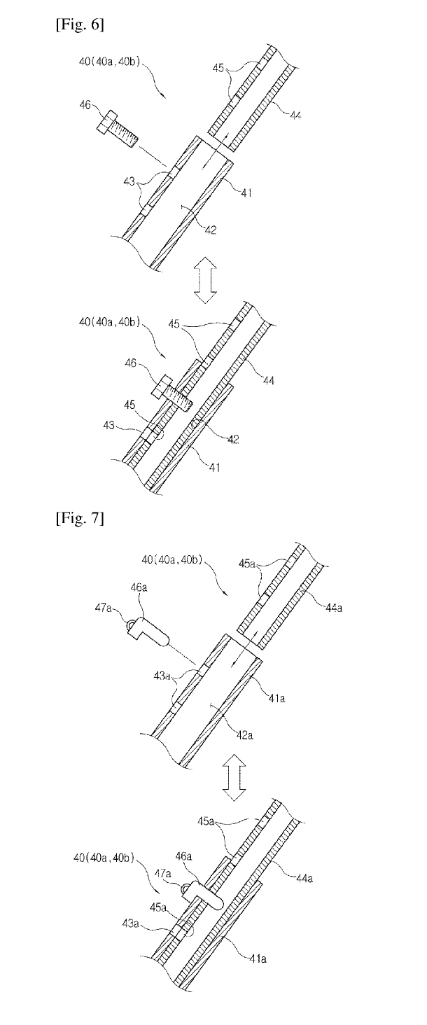

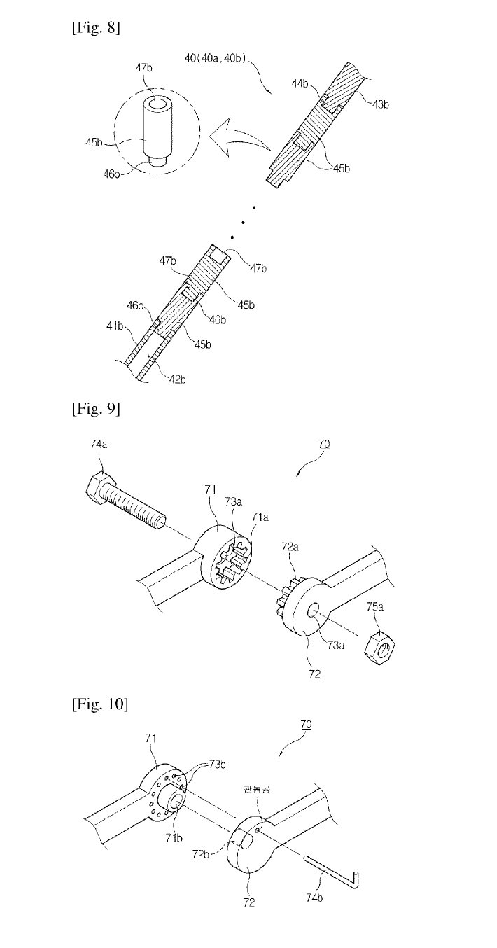

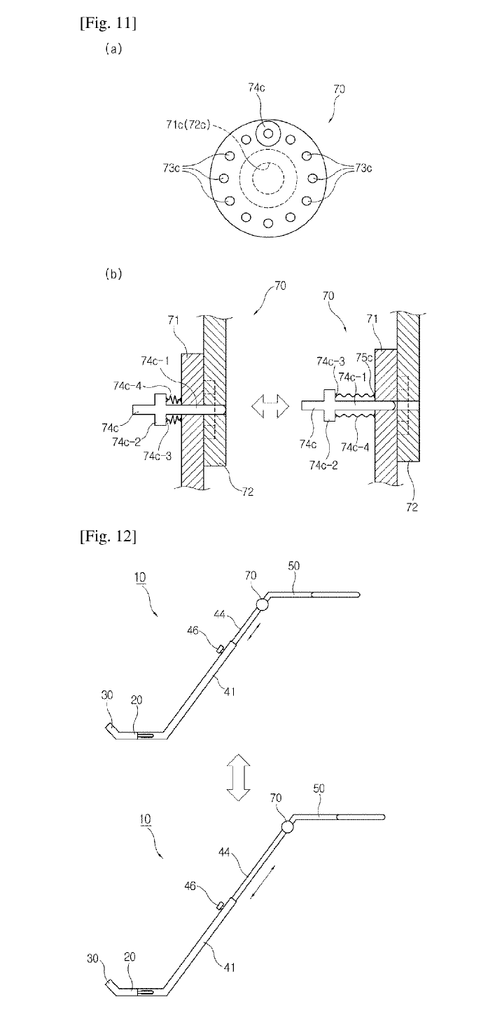

Hereinafter, according to the accompanying drawings, FIG. 1 is a perspective view showing a surgical instrument for an iliac vein surgery according to the present invention, FIGS. 2 and 3 are views of another embodiment of a surgical instrument for an iliac vein surgery according to the present invention, FIGS. 4 and 5 are views of another embodiment of a surgical instrument for an iliac vein surgery according to the present invention, FIGS. 6 to 8 are views showing a height control part of a surgical instrument for an iliac vein surgery according to the present invention, and FIGS. 9 to 11 are views showing a rotating part of a surgical instrument for an iliac vein surgery according to the present invention.

As shown in FIG. 1, a surgical instrument (10) for the iliac vein surgery according to the present invention includes a pressurizing part (20) holding an iliac vein, an inclined part (30) disposed at one end of the pressurizing part (20) to be inclined with respect to the pressurizing part (20), a height control part (40) disposed at the other end of the pressurizing part (20), which is opposite to the one end of the pressurizing part (20), to adjust a height thereof, and a handle part (50) disposed at an end of the height control part (50).

That is, the surgical instrument (10) for the iliac vein surgery shown in FIG. 1 is manufactured by integrally forming the pressurizing part (20), the inclined part (30), the height control part (40), and the handle part (50) with a member having a predetermined length.

In addition, as shown in FIGS. 2 and 3, the surgical instrument (10) for the iliac vein surgery of the present invention includes pressurizing parts (20a, 20b) holding the iliac vein, inclined parts (30a, 30b) respectively disposed at one ends of the pressurizing parts (20a, 20b) to be inclined with respect to the pressurizing parts (20a, 20b), height control parts (40, 40a, 40b) respectively disposed at the other ends of the pressurizing parts (20a, 20b), which are opposite to the one ends of the pressurizing parts (20a, 20b), to adjust a height thereof, and handle parts (50a, 50b) respectively disposed at ends of the height control parts (50a, 50b).

That is, the surgical instrument (10) for the iliac vein surgery shown in FIGS. 2 and 3 is manufactured by integrally forming the pressurizing parts (20a, 20b), the inclined parts (30a, 30b), the height control parts (40a, 40b), and the handle parts (50a, 50b) with two members, each having a predetermined length, and installing a rotating axis (60) at the height control parts (40a, 40b), and thus the surgical instrument (10) for the iliac vein surgery may be operated as scissors.

In addition, as shown in FIGS. 1 and 5, the surgical instrument (10) for the iliac vein surgery of the present invention includes a pair of pressurizing parts (20a, 20b) holding the iliac vein, height control parts (40a, 40b) respectively disposed at one ends of the pressurizing parts (20a, 20b) to adjust a height thereof, pressing parts (80a, 80b) respectively extending from the height control parts (40a, 40b), being curved, and being operated upon back and forth to allow the pressurizing parts (20a, 20b) to make or not to make contact with the height control parts (40a, 40b), and a handle part (50) disposed at ends of the pressing parts (80a, 80b).

That is, according to the surgical instrument (10) for the iliac vein surgery, a distance occurs between the pressurizing parts (20a, 20b) and the height control parts (40a, 40b) when the pressing parts (80a, 80b) are operated, and a designated portion may be held between and supported by the pressurizing parts (20a, 20b) and the height control parts (40a, 40b).

In this case, the surgical instrument (10) for the iliac vein surgery may be provided in a single unitary and individual unit or one pair of two units as shown in figures.

The pressurizing parts (20, 20a, 20b) placed at a lower portion of the surgical instrument (10) for the iliac vein surgery have a predetermined length to pressurize the iliac vein. In the present invention, the pressurizing parts (20, 20a, 20b) that are formed in a horizontal direction will be described in detail.

The inclined parts (30, 30a, 30b), which are disposed at the one ends of the pressurizing parts (20, 20a, 20b) and inclined with respect to the pressurizing parts (20, 20a, 20b), are inclined upward at a predetermined angle.

That is, the inclined parts (30, 30a, 30b) prevent organs from being damaged due to the ends of the pressurizing parts (20, 20a, 20b) and substantially simultaneously provide an operator with an insertion depth.

The height control parts (40, 40a, 40b) disposed at the other ends of the pressurizing parts (20, 20a, 20b), which are opposite to the one ends of the pressurizing parts (20, 20a, 20b), may be configured to the height thereof in accordance with the condition of patient. The height control parts (40, 40a, 40b) will be described with reference to FIGS. 6 to 8 as follows.

The height control parts (40, 40a, 40b) shown in FIG. 6 includes a lower control portion (41) having a predetermined height, including an accommodating space (42) defined therein, and including screw-engaging holes (43) formed therethrough and spaced apart from each other, an upper control portion (44) inserted into the accommodating space (42) and provided with screw-engaging recesses (45) formed therethrough and spaced apart from each other, and a fixing part (46) spiral-engaged with a corresponding screw-engaging hole of the screw-engaging holes (43) and a corresponding screw-engaging recess of the screw-engaging recesses (45).

In detail, the height control parts (40, 40a, 40b) may be manufactured and operated by inserting the upper control portion (44) into the accommodating space (42) of the lower control portion (41), controlling the height thereof depending on the condition of the patient, and spiral-engaging the fixing part (46) with the screw-engaging hole (43) and the screw-engaging recess (45).

In addition, the height control parts (40, 40a, 40b) shown in FIG. 7 includes a lower control portion (41a) having a predetermined height, including an accommodating space (42a) defined therein, and including lower thru-holes (43a) formed therethrough and spaced apart from each other, an upper control portion (44a) inserted into the accommodating space (42a) and provided with upper thru-holes (45a) formed therethrough and spaced apart from each other, and a fixing pin (46a) insertion-coupled with a corresponding lower thru-hole of the lower thru-holes (43a) and a corresponding upper thru-hole of the upper thru-holes (45a). The fixing pin (46a) includes a retaining ring (47a) disposed at an end of the fixing pin (46a) and bent.

In detail, the height control parts (40, 40a, 40b) may be manufactured and operated by inserting the upper control portion (44a) into the accommodating space (42a) of the lower control portion (41a), controlling the height thereof depending on the condition of the patient, and insertion-coupling the fixing pin (46a) with the lower thru-hole (43a) and the upper thru-hole (45a).

Then, the height control parts (40, 40a, 40b) shown in FIG. 8 includes a lower control portion (41b) including an accommodating space (42b) defined therein, an upper control portion (43b) separated from the lower control portion (41b) and including a coupling protrusion (44b) disposed at an end thereof, and a plurality of control members (45b) coupled between the lower control portion (41b) and the upper control portion (43b).

In detail, the height control parts (40, 40a, 40b) may be manufactured and operated by disposing the control members (45b) stacked on one another between the lower control portion (41b) and the upper control portion (43b) in consideration of the condition of the patient and coupling the control members (45b) with the lower control portion (41b) and the upper control portion (43b).

In this case, the control members 45b may be consecutively stacked on one another and coupled with each other using a coupling protrusion (46b) disposed at a lower end of each control member (45b) and a coupling recess (47b) disposed at an upper end of each control member (45b).

The handle part (50) disposed at the end of the height control part 40 has a predetermined length and includes a circular ring (52) disposed at an end of the handle part (50). The shape of the circular ring (52) should not be limited to the circular shape.

Then, a rotating part (70) is selectively equipped with the height control parts (40, 40a, 40b) or the handle parts (50, 50a, 50b) to rotate the handle parts (50, 50a, 50b) according to the condition of the patient, and thus the surgical instrument (10) may make contact with the patient's body. The rotating part 70 will be described with reference to FIGS. 9 to 11 as follows. In this case, the height control parts (40, 40a, 40b) or the handle parts (50, 50a, 50b) with which the rotating part (70) is equipped include a first separating part (71) and a second separating part (72) separated from the first separating part (71).

The rotating part (70) shown in FIG. 9 includes a rotation coupling hole (71a) formed in the first separating part (71) to have a gear shape and a rotation protrusion (72a) formed in the second separating part (72) to have a gear shape. The rotation protrusion (72a) is coupled to the rotation coupling hole (71a).

In detail, the rotating part (70) may be obtained and operated by inserting the rotation protrusion (72a) of the second separating part (72) into the rotation coupling hole (71a) of the first separating part (71) after an angle of the rotation protrusion (72a) is adjusted.

In this case, a thru-hole (73a) is formed through a center portion of each of the first and second separating parts (71, 72), a fixing bolt (74a) is inserted into the thru-hole (73a) at one side, and a fixing nut (75a) is engaged with the fixing bolt (74a) at the other side opposite to the one side.

In addition, the rotating part (70) shown in FIG. 10 includes an axis coupling hole (71b) and an axis coupling protrusion (72b) respectively formed through centers of the first separating part (71) and the second separating part (72) for the insertion-coupling between the first and second separating parts (71, 72). Pin coupling holes (73b) are formed through an end of the first separating part (71) to be spaced apart from each other along a circumference of the first separating part (71) and a rotation fixing pin (74b), of which one end thereof is bent, is insertion-coupled to the pin coupling holes (73b).

In detail, the rotating part (70) may be obtained and operated by insertion-coupling the second separating part (72) with the first separating part (71), rotating the first separating part (71) or the second separating part (72), and insertion-coupling the rotation fixing pin (74b) to the pin coupling holes (73b).

Then, the rotating part (70) shown in FIG. 11 includes an axis coupling hole (71c) and an axis coupling protrusion (72c) respectively formed through centers of the first separating part (71) and the second separating part (72) for the insertion-coupling between the first and second separating parts (71, 72). Insertion-coupling recesses (73c) are formed through an end of the first separating part (71) to be spaced apart from each other along a circumference of the first separating part (71) and an insertion-fixing member (74c) formed along an outer surface of the second separating part (72). The insertion-fixing member (74c) moves in a horizontal direction be insertion-coupled to the insertion-coupling recesses (73c) of the first separating part (71).

In addition, the insertion-fixing member (74c) includes a horizontal moving member (74c-1), to which a vertical plate (74c-2) inserted into or discharged from the insertion-coupling recess (73c) and including a spring-shaped ring (74c-3) in an intermediate portion of the insertion-fixing member (74c) is coupled, and a compression spring (74c-4) in which one end thereof is fixed to the spring-shaped ring (74c-3) and the other end thereof is fixed to a spring-shaped ring (75c) of the second separating part (72).

In detail, when the horizontal moving member (74c-1) is released after controlling the position of the first separating part (71) or the second separating part (72) in the state where the horizontal moving member (74c-1) equipped with the second separating part (72) is pulled outwardly, the horizontal moving member (74c-1) moves to the first separating part (71) due to elasticity of the compression spring (74c-4), and thus the first separating part (71) is coupled and fixed to the second separating part (72).

Hereinafter, an exemplary embodiment of the surgical instrument for the iliac vein surgery, which has the above-mentioned structure, will be described.

The pressurizing part (20) is formed at the one end of the member having the predetermined length, the inclined part (30) is formed at the one end of the pressurizing part (20) to be inclined with respect to the pressurizing part (20) at the predetermined angle, and then the handle part (50) having the circular ring (52) is formed at the other end of the pressurizing part (20), which is opposite to the one end of the pressurizing part (20).

Then, the pressurizing part (20) and the handle part (50) are separated from each other, and the height control part (40) is manufactured to include the lower control portion (41) having the predetermined height, including the accommodating space (42) defined therein, and including the screw-engaging holes (43) formed therethrough and spaced apart from each other, the upper control portion (44) inserted into the accommodating space (42) and provided with screw-engaging recesses (45) formed therethrough and spaced apart from each other, and the fixing part (46) spiral-engaged with the screw-engaging holes (43) and the screw-engaging recesses (45).

That is, the lower control portion (41) is formed at the end of the pressurizing part (20) and the upper control portion (44) is formed at the end of the handle part (50).

Then, the rotating part (70) is installed at the end of the height control part (40) or the handle part (50) to completely assemble the surgical instrument (10) for the Iliac vein surgery. To this end, the rotating part (70) includes the first and second separating parts (71, 72) separated from each other. The rotation coupling hole (71a) having the gear shape is formed in the first separating part (71), and thus the thru-hole (73a) is formed through the center portion of the first separating part (71). The rotation protrusion (72a) having the gear shape is protrusion-formed in the second separating part (72), and thus the thru-hole (73a) is formed through the center portion of the second separating part (72). The fixing bolt (74a) is inserted into the thru-hole (73a) at one side, and the fixing nut (75a) is engaged with the fixing bolt (74a) at the other side opposite to the one side.

Here, the assembling order of the surgical instrument for the iliac vein surgery should not be limited to the above-mentioned order.

Hereinafter, the state of use of the surgical instrument for the iliac vein surgery will be described.

First, the upper control portion (44) is inserted into the lower control portion (41) of the height control part (40), the upper control portion 44 is controlled to adjust the height of the surgical instrument in accordance with the condition of the patient, and then the fixing part (46) is spiral-engaged with the screw-engaging hole (43) and the screw-engaging recess (45).

Then, the rotating part (70) equipped with the height control part (40) or the handle part (50) is operated to control the angle of the handle part (50).

That is, the rotation protrusion (72a) of the second separating part (72) is inserted into the rotation coupling hole (71a) of the first separating part (71) after the angle of the rotation protrusion (72a) is adjusted with respect to the first separating part (71). Then, the fixing bolt (74a) is inserted into the thru-hole (73a) formed through each of the first and second separating parts (71, 72) and the fixing nut (75a) is engaged with the fixing bolt (74a).

Next, in the case where the surgical instrument (10) for the iliac vein surgery is used to perform anastomosis during the iliac vein surgery, the operator may pressurize the iliac vein using the pressurizing part (20) while gripping the handle part (50) of the surgical instrument (10) for the iliac vein surgery.

### INDUSTRIAL APPLICABILITY

Since the handle part (50) is in close contact with the patient's body as possible, the interference between the surgical instrument and the operator's hands may be minimized.

While the surgical instrument for the iliac vein surgery of the present disclosure has been shown and described with reference to various embodiments thereof, it will be understood by those skilled in the art that various changes in form and details may be made therein without departing from the spirit and scope of the present disclosure as defined by the appended claims and their equivalents.

1. A surgical instrument for an iliac vein surgery, comprising:
a pressurizing part for fixing an iliac vein;
an inclined part disposed at an end of one side of the pressurizing part and inclined with respect to the pressurizing part;
a height control part disposed at an end of an opposite side to the one side of the pressurizing part to control a height thereof; and
a handle part disposed at an end of the height control part.

2. A surgical instrument for an iliac vein surgery, comprising:
two pressurizing parts for fixing an iliac vein;
an inclined part disposed at each end of one side of the pressurizing parts and inclined with respect to the pressurizing parts;
a height control part disposed at each end of an opposite side to the one side of the pressurizing parts to control a height thereof;
a handle part disposed at an end of the height control part; and
a rotation axis equipped with a contact portion of the height control part.

3. A surgical instrument for an iliac vein surgery, comprising:
a pair of pressurizing parts for fixing an iliac vein;
a height control part disposed at each end of one side of the pressurizing parts to control a height thereof;
a pressing part extending from the height control part, being curved, and being operated upon back and forth to allow the pressurizing part to make or not to make contact with the height control part; and
a handle part disposed at an ends of the pressing part.

4. The surgical instrument of claim 1, wherein the height control part comprises:
a lower control portion having a predetermined height, comprising an accommodating space defined therein, and comprising screw-engaging holes formed therethrough and spaced apart from each other;
an upper control portion inserted into the accommodating space and provided with screw-engaging recesses formed therethrough and spaced apart from each other; and
a fixing part spiral-engaged with the screw-engaging holes and the screw-engaging recesses.

5. The surgical instrument of claim 1, wherein the height control part comprises:
a lower control portion having a predetermined height, comprising an accommodating space defined therein, and comprising lower thru-holes formed therethrough and spaced apart from each other;
an upper control portion inserted into the accommodating space and provided with upper thru-holes formed therethrough and spaced apart from each other; and
a fixing pin insertion-coupled with the lower thru-holes and the upper thru-holes and comprising a retaining ring disposed at an end of the fixing pin and bent.

6. The surgical instrument of claim 1, wherein the height control part comprises:
a lower control portion comprising an accommodating space defined therein;
an upper control portion separated from the lower control portion and comprising a coupling protrusion disposed at an end thereof; and
a plurality of control members coupled between the lower control portion and the upper control portion and comprising a coupling protrusion disposed at a lower end of each control member and a coupling recess disposed at an upper end of each control member to be insertion-coupled with each other.

7. The surgical instrument of claim 1, wherein the handle part has a predetermined length and comprises a circular ring disposed at an end thereof.

8. The surgical instrument of claim 1, wherein a rotating part is equipped with the height control part or the handle part, and the height control part or the handle part, with which the rotating part is equipped, comprises a first separating part and a second separating part separated from the first separating part.

9. The surgical instrument of claim 8, wherein the rotating part comprises a rotation coupling hole formed in the first separating part and having a gear shape and a rotation protrusion protrusion-formed in the second separating part having a gear shape.

10. The surgical instrument of claim 9, wherein a thru-hole is formed through a center portion of each of the first and second separating parts, a fixing bolt is inserted into the thru-hole at one side of the thru-hole, and a fixing nut is coupled with the fixing bolt at an opposite side to the one side.

11. The surgical instrument of claim 8, wherein the rotating part comprises an axis coupling hole and an axis coupling protrusion respectively formed through centers of the first separating part and the second separating part for the insertion-coupling between the first and second separating parts, pin coupling holes formed through an end of the first separating part to be spaced apart from each other along a circumference of the first separating part, and a rotation fixing pin, of which one end thereof is bent, insertion-coupled to the pin coupling holes.

12. The surgical instrument of claim 8, wherein the rotating part comprises an axis coupling hole and an axis coupling protrusion respectively formed through centers of the first separating part and the second separating part for the insertion-coupling between the first and second separating parts, insertion-coupling recesses formed through an end of the first separating part to he spaced apart from each other along a circumference of the first separating part, and an insertion-fixing member formed along an outer surface of the second separating part, and the insertion-fixing member moves in a horizontal direction to be insertion-coupled to the insertion-coupling recesses of the first separating part.

13. The surgical instrument of claim 12, wherein the insertion-fixing member comprises a horizontal moving member, to which a vertical plate inserted into or discharged from the insertion-coupling recess and comprising a spring-shaped ring in an intermediate portion of the insertion-fixing member is coupled, and a compression spring in which one end thereof is fixed to the spring-shaped ring and the other end thereof is fixed to a spring-shaped ring of the second separating part.

14. The surgical instrument of claim 2, wherein the height control part comprises:
a lower control portion having a predetermined height, comprising an accommodating space defined therein, and comprising screw-engaging holes formed therethrough and spaced apart from each other;
an upper control portion inserted into the accommodating space and provided with screw-engaging recesses formed therethrough and spaced apart from each other; and
a fixing part spiral-engaged with the screw-engaging holes and the screw-engaging recesses.

15. The surgical instrument of claim 3, wherein the height control part comprises:
a lower control portion having a predetermined height, comprising an accommodating space defined therein, and comprising screw-engaging holes formed therethrough and spaced apart from each other;
an upper control portion inserted into the accommodating space and provided with screw-engaging recesses formed therethrough and spaced apart from each other; and
a fixing part spiral-engaged with the screw-engaging holes and the screw-engaging recesses.

16. The surgical instrument of claim 2, wherein the height control part comprises:
a lower control portion having a predetermined height, comprising an accommodating space defined therein, and comprising lower thru-holes formed therethrough and spaced apart from each other;
an upper control portion inserted into the accommodating space and provided with upper thru-holes formed therethrough and spaced apart from each other; and
a fixing pin insertion-coupled with the lower thru-holes and the upper thru-holes and comprising a retaining ring disposed at an end of the fixing pin and bent.

17. The surgical instrument of claim 3, wherein the height control part comprises:
a lower control portion having a predetermined height, comprising an accommodating space defined therein, and comprising lower thru-holes formed therethrough and spaced apart from each other;
an upper control portion inserted into the accommodating space and provided with upper thru-holes formed therethrough and spaced apart from each other; and
a fixing pin insertion-coupled with the lower thru-holes and the upper thru-holes and comprising a retaining ring disposed at an end of the fixing pin and bent.

18. The surgical instrument of claim 2, wherein the height control part comprises:
a lower control portion comprising an accommodating space defined therein;
an upper control portion separated from the lower control portion and comprising a coupling protrusion disposed at an end thereof; and
a plurality of control members coupled between the lower control portion and the upper control portion and comprising a coupling protrusion disposed at a lower end of each control member and a coupling recess disposed at an upper end of each control member to be insertion-coupled with each other.

19. The surgical instrument of claim 3, wherein the height control part comprises:
a lower control portion comprising an accommodating space defined therein;
an upper control portion separated from the lower control portion and comprising a coupling protrusion disposed at an end thereof; and
a plurality of control members coupled between the lower control portion and the upper control portion and comprising a coupling protrusion disposed at a lower end of each control member and a coupling recess disposed at an upper end of each control member to be insertion-coupled with each other.

20. The surgical instrument of claim 2, wherein the handle part has a predetermined length and comprises a circular ring disposed at an end thereof.
